(19) United States
(12) Patent Application Publication
(10) Pub. No.: US 2016/0039871 A1
(43) Pub. Date: Feb. 11, 2016

(54) NOVEL FORMS OF TELAPREVIR

(71) Applicant: SANDOZ AG, Basel (CH)

(72) Inventors: Marijan Stefinovic, Kundl (AT); Christoph Langes, Innsbruck (AT); Ulrich Griesser, Innsbruck (AT)

(21) Appl. No.: 14/652,816

(22) PCT Filed: Dec. 20, 2013
(86) PCT No.: PCT/EP2013/077652

§ 371 Date: Jun. 17, 2015

Related U.S. Application Data
(60) Provisional application No. 61/740,841, filed on Dec. 21, 2012.

(30) Foreign Application Priority Data
Dec. 21, 2012 (EP) 12198875.2

(51) Int. Cl. C07K 5/103

(52) U.S. Cl. CPC C07K 5/1008

(57) ABSTRACT

The invention relates to an amorphous form of telaprevir, its preparation via novel crystalline Form C of telaprevir (also referred to as “crystalline Form C” or “Form C”), and telaprevir compositions comprising said amorphous form and Form C. Furthermore, the present invention relates to the use of said amorphous telaprevir, telaprevir composition and Form C of telaprevir for the preparation of medicaments such as anti-hepatitis C medicaments. Moreover, the present invention relates to pharmaceutical compositions and dosage forms comprising a pharmaceutically effective amount of said novel forms for use in treating patients suffering from hepatitis C virus.

The invention relates to an amorphous form of telaprevir, its preparation via novel crystalline Form C of telaprevir (also referred to as “crystalline Form C” or “Form C”), and telaprevir compositions comprising said amorphous form and Form C. Furthermore, the present invention relates to the use of said amorphous telaprevir, telaprevir composition and Form C of telaprevir for the preparation of medicaments such as anti-hepatitis C medicaments. Moreover, the present invention relates to pharmaceutical compositions and dosage forms comprising a pharmaceutically effective amount of said novel forms for use in treating patients suffering from e.g. hepatitis C virus.

### BACKGROUND PRIOR ART

Telaprevir, CAS no. 402957-28-2, PubChem no. (CID) 3010818, IUPAC name (1S,3aR,6aS)-2-[(2S)-2-[[(2S)-2-Cyclohexyl-2-(pyrazine-2-carbonylamino)acetyl]amino]-3,3-dimethylbutanoyl]-N-[(3S)-1-(cyclopropylamino)-1,2-dioxohexan-3-yl]-3,3a,4,5,6,6a-hexahydro-1H-cyclopenta[c]pyrrole-1-carboxamide (also named as VX-950) is the isomer of Formula 1 (which is the free base form)

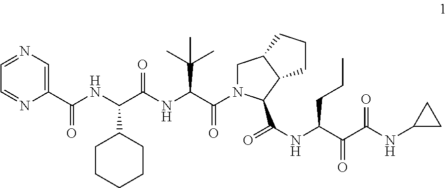 [SMILES: CCC[C@H](NC(=O)[C@@H]1[C@H]2CCC[C@H]2CN1C(=O)[C@@H](NC(=O)[C@@H](NC(=O)c1cnccn1)C1CCCCC1)C(C)(C)C)C(=O)C(=O)NC1CC1]

and is a protease inhibitor that can be used as antiviral drug. By way of example, telaprevir inhibits the hepatitis C virus NS3-4A serine protease. Any epimeric impurities related to Formula 1 can be determined by HPLC (High-performance liquid chromatography) or NMR (nuclear magnetic resonance spectroscopy).

Telaprevir is commercially available from various suppliers as Form A. Furthermore, different processes for the synthesis of telaprevir or certain salts are available. For example, WO 2007/022459 A2 and Turner et al. (Chemical Communications 2010, 46(42), 7918) disclose processes for the preparation of telaprevir. Telaprevir can also be prepared as described in the references cited in WO 2009/032198 A1.

Crystalline Form A of telaprevir is described by Kwong et al. (2011). However, this crystalline form suffers from a poor solubility in water of about 0.0045 mg/mL in water.

WO 2009/032198 A1, WO2007098270 A2 and WO 2008/106151 A2 describe co-crystals of telaprevir and co-crystal formers such as 2,5-dihydroxybenzoic acid and their preparation from e.g. acetonitrile and dichloromethane mixtures. Said co-crystals shall possess an improved dissolution and higher aqueous solubility. However there is a safety issue associated with the co-crystal formers.

US 2012/0083441 A1 discloses the preparation of solid dispersions of telaprevir and polymers such as HPMC (hydroxypropylmethylcellulose) or HPMCAS (hydroxypropylmethylcellulose acetate succinate) polymers and surfactants by spray drying. Spray drying on a commercial scale is a cost intensive investment and especially when solvents or solvent mixtures disclosed in the above patent application are used.

The “PriorArtDatabase” (ip.com) contains an anonymous publication (IPCOM000213558D) with the title “Amorphous (1S,3aR,6aS)-N-(1(S)-(2-(Cyclopropylamino)oxalyl)butyl)-2-(N-(pyrazin-2ylcarbonyl)-L-cyclohexylglycyl-3-methyl-Lvalyl)perhydrocyclopenta[c]pyrrole-1-carboxamide”. It discloses an amorphous form of telaprevir obtained by dissolving telaprevir in dichloromethane and then evaporating to dryness. The PXRD pattern in this document shows two or three very broad peaks which are in accordance with an amorphous substance. However in our hands the solvent content of this material was unacceptably high.

In view of the above disadvantages there is a need to prepare an amorphous form of telaprevir suitable for the manufacture of a dosage form avoiding the problems with the known amorphous forms and the solubility problems associated with the crystalline Form A of telaprevir.

### SUMMARY OF THE INVENTION

The invention particularly relates to neat amorphous telaprevir, meeting ICH guidelines for solvent content, methods of its preparation and its use in the preparation of finished dosage forms.

In another embodiment the invention relates to novel forms of telaprevir, namely crystalline Form C.

In another embodiment, the invention relates to the preparation of neat amorphous telaprevir, meeting ICH guidelines for solvent content from the novel Form C of telaprevir.

In a further aspect the invention relates to the use the neat amorphous form of the invention in the manufacture of a medicament comprising telaprevir.

In more detail, the invention relates to a process for the preparation of an amorphous form of telaprevir of Formula 1

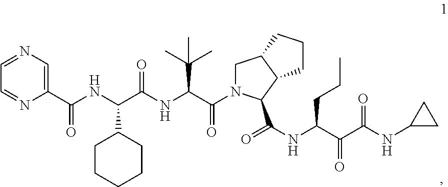 [SMILES: CCC[C@H](NC(=O)[C@@H]1[C@H]2CCC[C@H]2CN1C(=O)[C@@H](NC(=O)[C@@H](NC(=O)c1cnccn1)C1CCCCC1)C(C)(C)C)C(=O)C(=O)NC1CC1]

comprising the steps of:

(i) providing a crystalline Form C of telaprevir which is characterized by an X-ray powder diffraction pattern with peaks at 2-theta angles of 6.6±0.2 degrees 2theta, 7.0±0.2 degrees 2theta, 8.0±0.2 degrees 2theta, 8.9±0.2 degrees 2theta, 9.4±0.2 degrees 2theta, 17.6±0.2 degrees 2theta, when using Cu-Kα radiation, and

(ii) converting said crystalline Form C of telaprevir into said amorphous form of telaprevir.

The invention also refers to an amorphous form of telaprevir obtainable or obtained by the process according to the invention.

Furthermore, the invention relates to an amorphous form of telaprevir of Formula 1 consisting of

(i) the compound of Formula 1 having a purity of at least 95 HPLC area-%, calculated by excluding solvents, and

(ii) solvents;

wherein (I) dichloromethane is present as a solvent in said amorphous form in an amount of from 1 ppm to 600 ppm, (II) water is present as a solvent in an amount of from 0.01 wt.-% to 10 wt.-%, and (III) solvents other than dichloromethane and water are present in a total amount of from 0 ppm to 5000 ppm.

The invention also relates to a Telaprevir composition comprising

(A) amorphous telaprevir, preferably according to the invention, and

(B) crystalline Form C of telaprevir according to the invention;

wherein said telaprevir composition consists of

(i) the compound of Formula 1

having a purity of at least 95 HPLC area-%, calculated by excluding solvents, and

(ii) solvents;

wherein (I) dichloromethane is present as solvent in said telaprevir composition in an amount of from 1 ppm to 600 ppm, (II) water is present as solvent in an amount of from 0.01 wt.-% to 10 wt.-%, and (III) solvents other than dichloromethane and water are present in a total amount of from 0 ppm to 5000 ppm, preferably water is present in an amount of from 0.5 wt.-% to 4.2 wt.-% and solvents other than water are present in the aforementioned amounts.

Furthermore, the invention refers to a crystalline Form C of telaprevir of Formula 1 suitable as intermediate for the preparation of an amorphous form of telaprevir, characterized by an X-ray powder diffraction pattern with peaks at 2-theta angles of 6.6±0.2 degrees 2theta, 7.0±0.2 degrees 2theta, 8.0±0.2 degrees 2theta, 8.9±0.2 degrees 2theta, 9.4±0.2 degrees 2theta, 17.6±0.2 degrees 2theta, when using Cu-Kα radiation. It also refers to crystalline telaprevir form C obtainable or obtained by using a process as described herein.

The invention also refers to a process for the preparation of crystalline Form C of telaprevir, said method comprising the steps of:

(i) providing seed crystals of said crystalline Form C of telaprevir;

(ii) providing a highly concentrated residue by evaporation of solvent from a solution of telaprevir in dichloromethane at reduced pressure and at a temperature in the range of from −78° C. to below 5° C., until a semi-solid mass is formed, wherein said dichloromethane contains water in amounts up to the saturation of dichloromethane with water;

(iii) adding said seed crystals of step (i) to said highly concentrated residue of step (ii) to form a mixture; and

(iv) evaporating solvent from said mixture of step (iii) to obtain said crystalline Form C of telaprevir.

The invention also refers to a pharmaceutical composition comprising

(i) a telaprevir composition according to the invention,

(ii) an amorphous form of telaprevir according to the invention, or (

iii) crystalline Form C of telaprevir according to the invention;

in admixture with one or more pharmaceutically acceptable excipients.

The invention also refers to crystalline Form C of telaprevir described herein, the amorphous form of telaprevir described herein, or the telaprevir composition described herein for use as medicament, in particular for use in a method of treating viral infections, such as infections caused by hepatitis C virus.

It also refers to a pharmaceutical dosage form comprising (i) the crystalline Form C of telaprevir described herein, (ii) the amorphous form of telaprevir described herein, (iii) the telaprevir composition described herein, and/or (iv) the pharmaceutical composition described herein.

### LIST OF FIGURES

FIG. 1: FIG. 1 shows amorphous telaprevir prepared from Form C

FIG. 2: FIG. 2 shows a PXRD pattern of Form C.

FIG. 3: FIG. 3 shows an FTIR spectrum of Form C

FIG. 4: FIG. 4 shows a Dynamic Vapor Sorption Curve for Form C

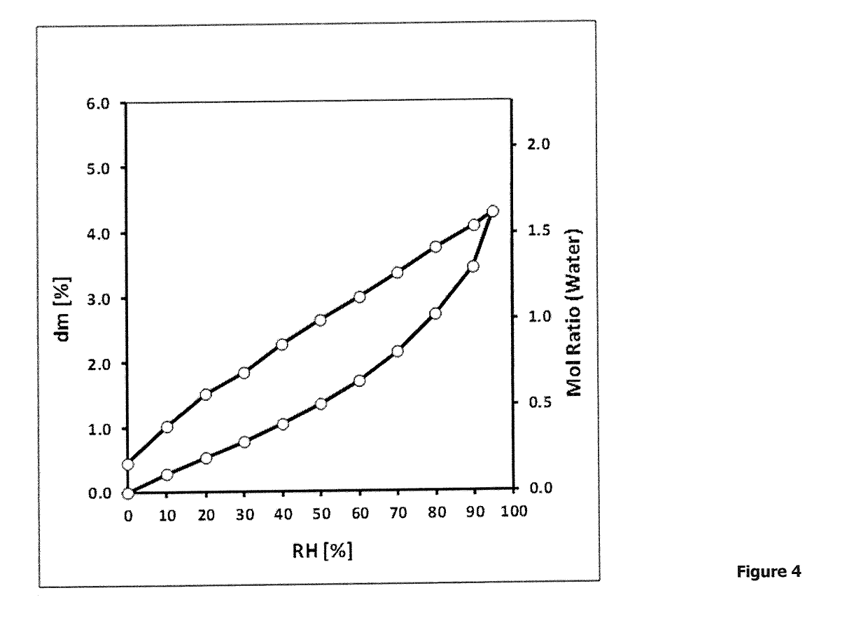

FIG. 5: FIG. 5 shows a DSC trace for Form C

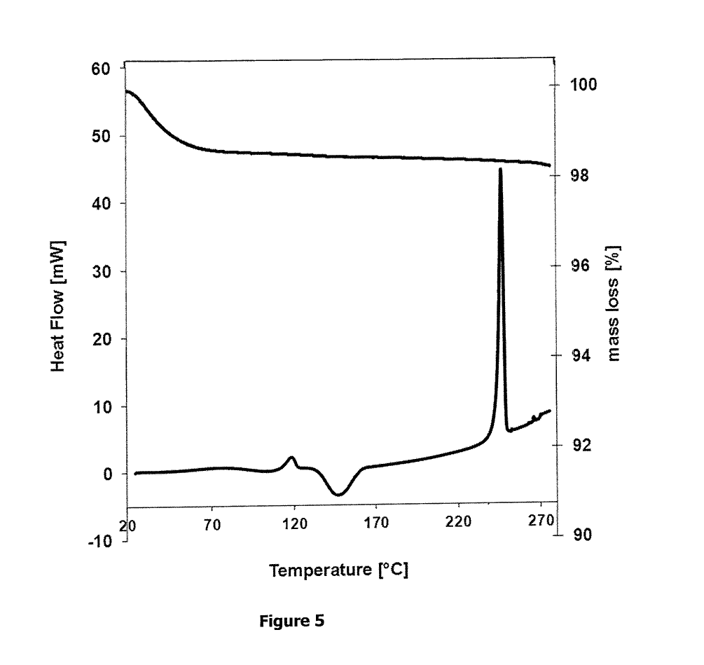

FIG. 6: FIG. 6 depicts the results of storing the amorphous phase at 46, 75, 84% RH for 22d. No reflections of any known crystalline form of telaprevir are shown.

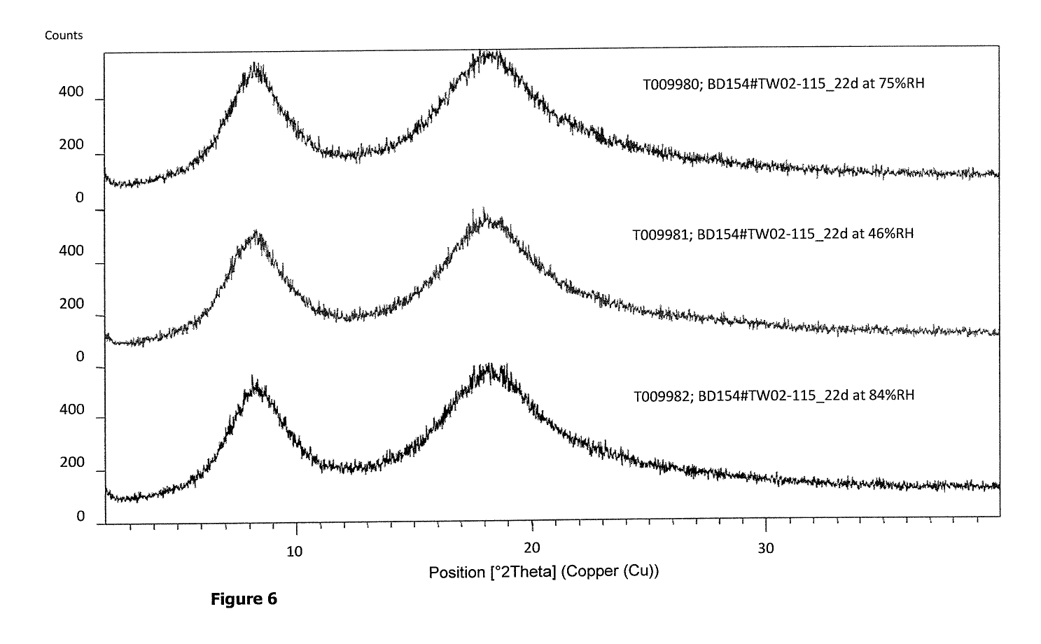

### DETAILED DESCRIPTION

It has unexpectedly been found that it is possible to provide a new valuable polymorphic form of telaprevir and its surprising use in the manufacture of compositions comprising neat substantially pure amorphous telaprevir meeting ICH guidelines for residual solvents.

Crystalline Form C of telaprevir according to the invention may provide several benefits over the known crystalline Form A and the known co-crystals mentioned above, since it for example is highly susceptible to grinding and pressure loosing its crystalline structure by converting to an amorphous state. The very mild conditions needed for the conversion from a crystalline state to an amorphous state preserves the chemical purity of the active pharmaceutical ingredient, e.g. no epimerization nor formation of degradation products is observed during this operation.

Furthermore, the content of dichloromethane in crystalline Form C is very low, e.g. lower than the ICH—International Conference on Harmonisation of Technical Requirements for the Registration of Pharmaceuticals for Human Use—limit of 600 ppm) as determined by GC, such as only 55 ppm. The low content of organic solvent of Form C passes to the low content of the neat amorphous telaprevir when e.g. Form C is e.g. ground e.g. by mild ball milling or even by gentle grinding with a mortar and pestle.

As is explained in the following, crystalline Form C of telaprevir could not be obtained by performing a routine screening for polymorphs. Telaprevir primarily exists in a single stable non-solvated form (Form A) obtainable from most common solvents.

Following the prior art (IPCOM000213558D), dissolving telaprevir in dichloromethane and then evaporating to dryness, in our hands also resulted in an amorphous form of telaprevir, but with an unacceptably high content of residual organic solvent, see Reference Example 2.

In addition, lyophilisation of a solution of telaprevir in tetrahydrofuran, a fairly low boiling solvent in which telaprevir is acceptably soluble, resulted in an amorphous telaprevir again with an unacceptably high solvent content.

Absolutely surprisingly, according to the present invention, Form C of telaprevir is obtainable from dichloromethane by using the process described herein.

The invention is further described with respect to the following items:

1 Process for the preparation of an amorphous form of telaprevir of Formula 1

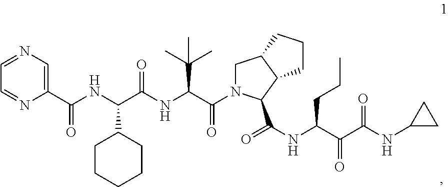 [SMILES: CCC[C@H](NC(=O)[C@@H]1[C@H]2CCC[C@H]2CN1C(=O)[C@@H](NC(=O)[C@@H](NC(=O)c1cnccn1)C1CCCCC1)C(C)(C)C)C(=O)C(=O)NC1CC1]

comprising the steps of:

(i) providing a crystalline Form C of telaprevir which is characterized by an X-ray powder diffraction pattern with peaks at 2-theta angles of about 6.6±0.2 degrees 2theta, 7.0±0.2 degrees 2theta, 8.0±0.2 degrees 2theta, 8.9±0.2 degrees 2theta, 9.4±0.2 degrees 2theta, 17.6±0.2 degrees 2theta, when using Cu-Kα radiation, and

(ii) converting said crystalline Form C of telaprevir into said amorphous form of telaprevir. Said crystalline Form C of telaprevir is further described in items 1.1 and 1.5-1.8 as well as items 5-5.2.

Form C of telaprevir surprisingly converts to amorphous Telaprevir on very mild grinding or under mild pressure, e.g. by grinding in a ball mill for a few minutes to several hours. E.g. 20 min to 24 h depending on the batch size and forces involved. The content of crystalline Form C decreases whereas the amount of amorphous telaprevir increases. The amount of crystalline form C can easily be monitored by PXRD analysis or IR analysis of the mixture.

Depending on the time and intensity of milling amorphous telaprevir of the invention is obtained.

The amorphization procedure may be performed on neat form C.

The neat substantially pure amorphous telaprevir meets current ICH guidelines for solvent content.

1.1 The process of item 1, wherein the crystalline Form C of telaprevir and/or the amorphous form of telaprevir consist of

(i) the compound of Formula 1 having a purity of at least 95 HPLC area-%, calculated by excluding solvents, and

(ii) solvents;

wherein (I) dichloromethane is present as a solvent in said amorphous form/Form C in an amount of from 1 ppm to 600 ppm, (II) water is present as a solvent in an amount of from 0.01 wt.-% to 10 wt.-%, and (III) solvents other than dichloromethane and water are present in a total amount of from 0 ppm to 5000 ppm. Further embodiments are described in the context of Form C and amorphous form of telaprevir below.

As used in the context of the present invention, the compound of Formula 1 has a purity of at least 95 HPLC area-%. Within the aforementioned range, the compound of Formula 1 can have a purity of at least 97 HPLC area-%. Within the aforementioned range, the compound of Formula 1 can have a purity of at least 98 HPLC area-%. Within the aforementioned range, the compound of Formula 1 can have a purity of at least 98.5 HPLC area-%. Within the aforementioned range, the compound of Formula 1 can have a purity of at least 99 HPLC area-%. Within the aforementioned range, the compound of Formula 1 can have a purity of at least 99.5 HPLC area-%. Within the aforementioned ranges, the compound of Formula 1 can have a purity with an upper limit of e.g. 99.8 HPLC area-%. Within the aforementioned ranges, the compound of Formula 1 can have a purity with an upper limit of 99.5 HPLC area-%. The purity can be 95 HPLC area-% to 99.9 HPLC area-%. The purity can be 97 HPLC area-% to 99.9 HPLC area-%. The purity can be 97 HPLC area-%, to 99 HPLC area-%. Within the meaning of the present invention, the compound of Formula 1 has a certain purity, wherein the remainder are impurities. Impurities are components other than telaprevir of Formula 1, excluding solvents, for example isomers related to Formula 1 and impurities resulting from the manufacture of the compound of Formula 1 such as intermediate products or reactants. Such impurities are typically contained in any pharmaceutically active ingredient.

Within the meaning of the present invention, the term “solvent” refers to water and further solvents such as organic solvents. Preferred solvent contents are described below, e.g. in items 3-3.1, and 4.5, which solvent contents can be present in the amorphous form and/or the crystalline Form C of the present invention.

Herein, purity levels, including chemical and chiral impurities, can be determined by HPLC.

The crystalline Form C of telaprevir is used in amounts suitable for the process applied for converting said Form C into the amorphous form. For example, if the crystalline Form C of telaprevir is converted by applying a grinding process, it is used in amounts commonly used such a grinding process and the grinding device, respectively.

1.2 The process of item 1 or 1.1, wherein converting said crystalline Form C of telaprevir into said amorphous form of teleprevir is not performed by dissolving/adding solvents or melting said crystalline Form C of telaprevir and is not performed at temperatures above 50° C. Preferably, it is performed by grinding at a temperature in the range of from 0° C. to 50° C., preferably 20° C. to 50° C. to avoid racemization of telaprevir.

1.3 The process of any of items 1-1.2, wherein the grinding process is performed until the PXRD does not show any distinct peaks anymore or wherein the grinding process is performed such that the desired percentage of amorphous form is obtained. The percentage of amorphous form can be determined as described in item 4.1.

The grinding process can be performed until the PXRD does not show any distinct peaks anymore and a completely amorphous telaprevir is obtained. For determining suitable grinding conditions, a skilled person can vary the applied pressure and shear forces until a conversion can be achieved as can be assayed by PXRD.

The formation of the amorphous form of telaprevir can be assayed by taking aliquots of the grinded material and subjecting same to PXRD using Cu-Kα radiation and conditions suitable for the detection of crystalline Form C of telaprevir, preferably by applying the devices and conditions described herein. The wavelength of the Cu-Kα radiation used herein is preferably λ=1.5406 Angstrom.

1.4 The process of any of items 1-1.3, wherein the devices used for converting said crystalline Form C of telaprevir into said amorphous form of telaprevir are mortar and pestle; ball mill, such as planetary ball mill; or grinding rolls.

Due to the pressure, and shear forces applied onto the crystals of Form C of telaprevir, for example by grinding/milling the crystals, the crystalline Form C transforms into the amorphous form.

1.5 The process of any of items 1-1.4, wherein said crystalline Form C of telaprevir has an X-ray powder diffraction pattern which does not show peaks of other crystal forms, and/or wherein said amorphous form of telaprevir has an X-ray powder diffraction pattern, when using Cu-Kα radiation, which does not show distinct peaks.

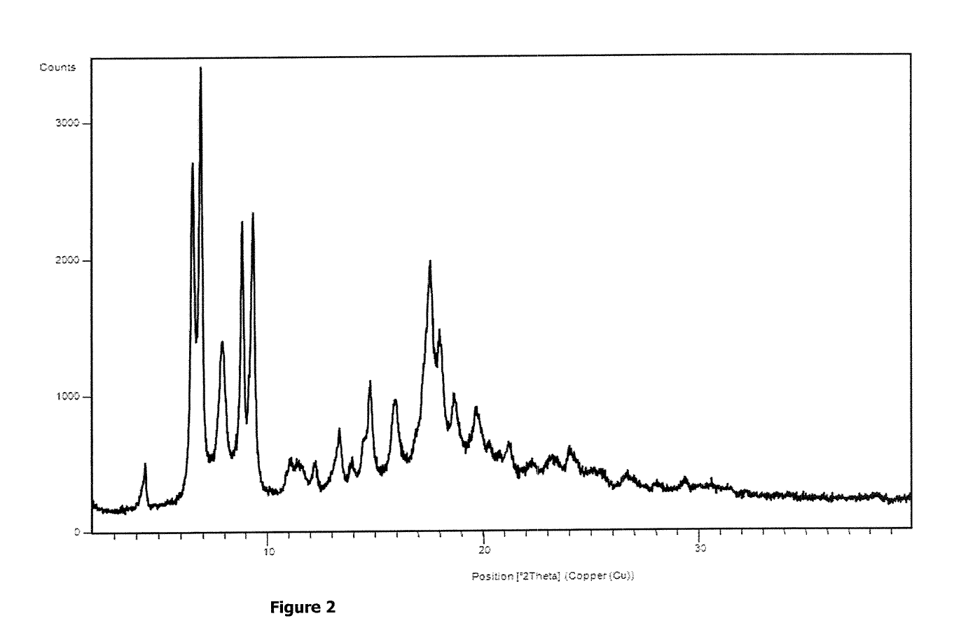

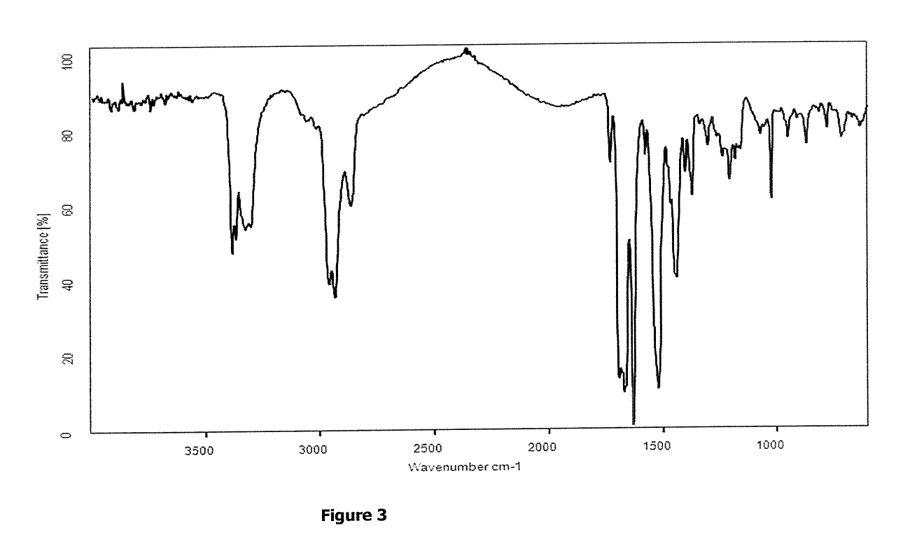

The crystalline Form C of telaprevir according to the invention is specifically characterized by an PXRD (powder X-ray diffraction) pattern substantially in accordance to FIG. 2. The crystalline Form C of telaprevir according to the invention is specifically characterized by a FTIR (Fourier transform infrared spectroscopy) spectrum substantially in accordance to FIG. 3.

1.6 The process of any of items 1-1.5, wherein said crystalline Form C of telaprevir and/or the amorphous form of telaprevir contains dichloromethane in an amount of from 5 ppm to 600 ppm, as determined by gas chromatography (GC).

The GC method suitable for measuring e.g. solvent contents is described below:

Device: GC HP6890 (Saulakopf)+HS: agilent G1888; Column: DB624/14; 30 m×0.32 mm*1.8 μm; Part. No. 123-1334; Ser. USB454863H; mobile phase: helium; He-flow: 3 ml/min; Gas saver: ON; T1: 40° C.(3 min); Rate: 10° C./min; T2: 200° C.(0 min); Detector: FID 250° C.; injector-temp.: 220° C.; hydrogen: 30 ml/min; air: 300 ml/min; range: 0; attn.: 0; split ratio: 1/10; HS-method: bath (oven): 80° C.; 1 ml loop: 120° C.; Tr. line: 150° C.; Equil. time: 20 min; inj. Time: 0.5 min; pressurization time=0.20 min; loop fill time=0.20 min; loop equil. time=0.05 min; shaker: low level; cycletime 28 min.

1.7 The process of any of items 1-1.6, wherein the crystalline Form C of telaprevir and/or the amorphous form of telaprevir has a water content of up to 4.2 wt.-%, such as a water content in the range of from 0.5 wt.-% to 4.2 wt-%, corresponding to 5000 ppm to 42000 ppm. The water content can be determined by TGA (thermogravimetric analysis), or preferably by Karl Fischer as described in K. Fischer Angew. Chemie 48, 394 (1935) or in “Die Tablette, Handbuch der Entwicklung, Herstellung and Qualitätssicherung”, Annette Bauer-Brandl, Wolfgang A. Ritschel, third edition, 2012, German language, Editio Cantor Verlag Aulendorf, Chapter 4.8.11, page 419.

1.8 The process of any of items 1-1.7, wherein said crystalline Form C of telaprevir is characterized by an infrared spectrum with peaks at wavenumbers of 3306.4±2 cm⁻¹, 2931.7±2 cm⁻¹, 2866.6±2 cm⁻¹, 1656.2±2 cm⁻¹, 1618.5±2 cm⁻¹, 1513.8±2 cm⁻¹, 1438.5±2 cm⁻¹, 1400.7±2 cm⁻¹, 1368.4±2 cm⁻¹, 1231.1±2 cm⁻¹, 1168.9±2 cm⁻¹, 1049.8±2 cm⁻¹, 1020.0±2 cm⁻¹, 944.2±2 cm⁻¹, 865.6, 775.3±2 cm⁻¹.

The process of the invention is suitable for preparing telaprevir compositions disclosed herein comprising the amorphous form of telaprevir according to the invention as well as Form C of telaprevir according to the invention. In order to provide that Form C of telaprevir is not completely converted into the amorphous form, the process of converting Form C of telaprevir can be terminated before the complete amount of Form C of telaprevir is converted, as can be monitored by PXRD which allows identifying Form C of telaprevir.

2 Amorphous, solid, form of telaprevir obtainable or obtained by the process according to any of items 1-1.8.

3 Amorphous, solid, form of telaprevir of Formula 1 (see above)

consisting of (i) the compound of Formula 1 having a purity of at least 95 HPLC area-%, excluding solvents, and (ii) solvents; wherein the solvent dichloromethane is present in said amorphous form in an amount of from 1 ppm to 600 ppm, water is present in an amount of 0.01-10 wt.-%, and solvents other than dichloromethane and water are present in a total amount of from 0 ppm to 5000 ppm. Within the aforementioned range, solvents other than dichloromethane and water can be present in an amount of from 0 ppm to 4000 ppm. Within the aforementioned range, solvents other than dichloromethane and water can be present in an amount of from 0 ppm to 3000 ppm. Within the aforementioned range, solvents other than dichloromethane and water can be present in an amount of from 0 ppm to 2000 ppm. Within the aforementioned range, solvents other than dichloromethane and water can be present in an amount of from 0 ppm to 1000 ppm. Within the aforementioned range, solvents other than dichloromethane and water can be present in an amount of from 0 ppm to 500 ppm. Water may preferably be comprised in amounts of from 0.5 wt.-% to 4.2 wt.-%. Preferably, solvents are water and organic solvents only.

The compound of Formula 1 can also have a purity as defined in item 1.1 above, for example a purity in the range of from 95 HPLC area-% to 99.9 HPLC area-%.

3.1 The amorphous form of telaprevir of item 3 which conforms with the ICH (INTERNATIONAL CONFERENCE ON HARMONISATION OF TECHNICAL REQUIREMENTS FOR REGISTRATION OF PHARMACEUTICALS FOR HUMAN USE) residual solvent guidelines, “GUIDELINE FOR RESIDUAL SOLVENTS Q3C(R5)”, Step 4 version dated 4 Feb. 2011.

Specifically, in accordance with the ICH solvent guidelines, the amorphous form of telaprevir contains the below organic solvents in maximum amounts as follows, preferably with a total content of solvents other than water and dichloromethane as defined above, e.g. 0-5000 ppm: carbon tetrachloride 4 ppm, benzene 2 ppm, 1,2-dichloroethane 5 ppm, 1,2-dichloroethane 5 ppm, 1,1,1-trichloroethane 1500 ppm, acetonitrile 410 ppm, chlorobenzene 360 ppm, chloroform 60 ppm, cumene 70 ppm, cyclohexane 3880 ppm, 1,2-dichloroethene 1870 ppm, dichloromethane 600 ppm, 1,2-dimethoxyethane 100 ppm, N,N-dimethylacetamide 1090 ppm, N,N-dimethylformamide 880 ppm, 1,4-dioxane 380 ppm, 2-ethoxyethanol 160 ppm, ethyleneglycol 620 ppm, formamide 220 ppm, hexane 290 ppm, methanol 3000 ppm, 2-methoxyethanol 50 ppm, methylbutyl ketone 50 ppm, methylcyclohexane 1180 ppm, N-methylpyrrolidone 530 ppm, nitromethane 50 ppm, pyridine 200 ppm, sulfolane 160 ppm, tetrahydrofuran 720 ppm, tetralin 100 ppm, toluene 890 ppm, 1,1,2-trichloroethene 80 ppm, xylene 2170 ppm. The following organic solvents should be limited to 5000 ppm: acetic acid, heptanes, acetone, isobutyl acetate, anisole, isopropyl acetate, 1-butanol methyl acetate, 2-butanol, 3-methyl-1-butanol, butyl acetate, methylethyl ketone, tert-butylmethyl ether, methylisobutyl ketone, dimethyl sulfoxide, 2-methyl-1-propanol, ethanol, pentane, ethyl acetate, 1-pentanol, ethyl ether, 1-propanol, ethyl formate, 2-propanol, formic acid, propyl acetate. Other organic solvents should each be limited to 500 ppm, 250 ppm, 100 ppm, 50 ppm, preferably 0 ppm. Further dichloromethane limits are defined in item 4.5.

3.2 The amorphous form of telaprevir of item 3 or 3.1 having an X-ray powder diffraction pattern, when using Cu-Kα radiation, which shows a halo. A halo can vary in shape and position because it reflects some order in the amorphous state.

3.3 The amorphous form of telaprevir of any of items 3-3.2 having an X-ray powder diffraction pattern, when using Cu-Kα radiation, which does not show distinct peaks. A crystalline phase has distinct peaks which are well defined in terms of height and width.

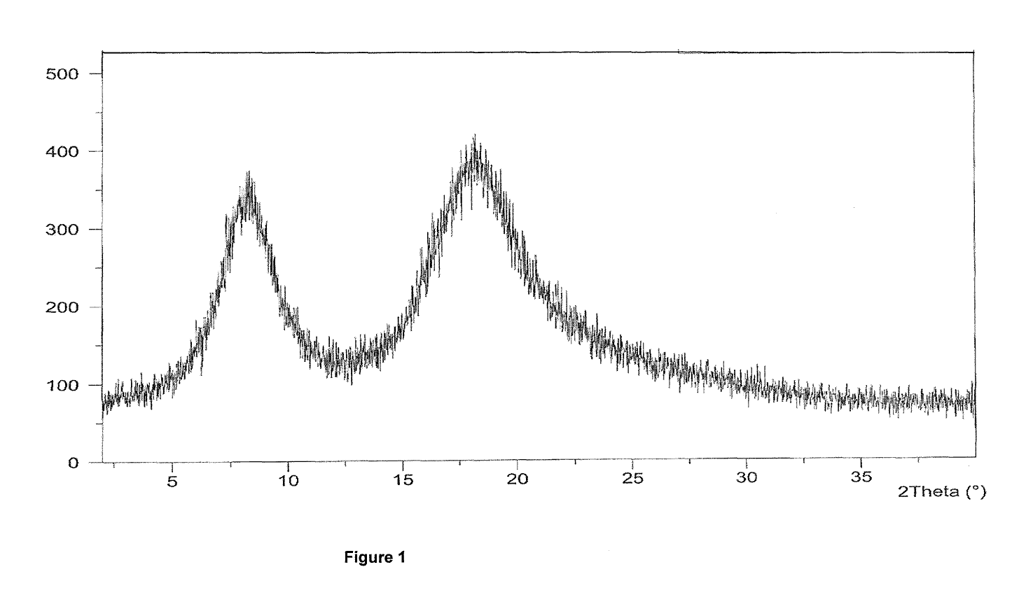

3.4 The amorphous form of telaprevir of any of items 3-3.3 can have a PXRD pattern which is essentially in accordance with FIG. 1. The present invention also refers to amorphous telaprevir only being characterized by having a PXRD pattern essentially in accordance with FIG. 1.

3.5 The amorphous form of telaprevir of any of items 3-3.4 may be stable when exposed to an equilibrium relative humidity of up to about 70%. A test for stability against humidity can be performed as follows: The sample is exposed to a relative humidity of 70% (+/−5%) at 50° C. (+/−2° C.) for 2 weeks (14 days). The expression “polymorphically stable” means that no conversion to a crystalline form occurs, as determined by PXRD. The present invention also refers to amorphous telaprevir only being characterized by having the aforementioned stability.

Thus, the amorphous telaprevir of the invention is polymorphically stable e.g. it does not convert to Form A when stored at about less than 70% e.g. about less than 60% relative humidity for an extended period of time e.g. for about two weeks. The amorphous telaprevir of the invention is also polymorphically stable in the pharmaceutical formulations described herein e.g. when the relative humidity of the above described mixtures respectively formulations is kept at a relative humidity of about less than 70% (+/−5%) e.g. less than 60% (+/−5%).

Furthermore, the amorphous telaprevir of the invention is stable on storage, either in pure form or when being present in a pharmaceutical composition or final dosage form. “Stable on storage” as defined herein means that even after storage for 180 days at low humidity (e.g. below 70%), and preferably even after storage for two years, the amorphous form of telaprevir is still present as substantially pure amorphous telaprevir without being converted to crystalline forms. Particularly stable compositions can be produced by avoiding humid conditions, such as high relative humidity of the air, during the formulation steps. Furthermore, humid conditions are to be avoided during storage of the amorphous telaprevir or the pharmaceutical composition or dosage form containing said amorphous telaprevir in order to preserve said amorphous form.

The equilibrium relative humidity of a sample is measured by determining the relative humidity in % in the air above a test sample, after establishment of a humidity equilibrium in a closed system at a constant temperature according to the following method: the equipment used is the commercially available measuring chamber Rotronic AW-VC comprising a hygrometer of the type BT-RS1. The test sample, is filled into a sampling dish which is placed into the measuring chamber which has been thermostatted to a temperature of 25+/−1° C., said chamber is subsequently closed and sealed. After establishment of an equilibrium of the relative humidity which state is typically shown by the disappearance of a trend indication, the value of the relative humidity in % is read from the hygrometer. Relative humidity is defined as the equilibrium relative humidity of the neat substantially pure amorphous telaprevir or pharmaceutical compositions thereof as measured as herein described.

3.6 The amorphous form of telaprevir of any of items 3-3.5 can be in the form of particles, wherein said particles preferably do not contain pharmaceutical excipients such as polymeric materials.

4 Telaprevir composition comprising/consisting of

(A) amorphous telaprevir, preferably according to any of items 2-3.6, and

(B) crystalline Form C of telaprevir as defined in any of the above or below items;

wherein said telaprevir composition consists of

(i) the compound of Formula 1

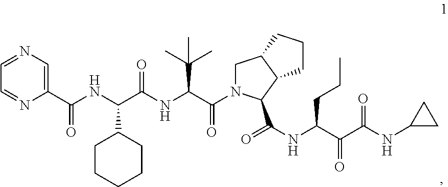 [SMILES: CCC[C@H](NC(=O)[C@@H]1[C@H]2CCC[C@H]2CN1C(=O)[C@@H](NC(=O)[C@@H](NC(=O)c1cnccn1)C1CCCCC1)C(C)(C)C)C(=O)C(=O)NC1CC1]

having a purity of at least 95 HPLC area-%, calculated by excluding solvents, and

(ii) solvents;

wherein (I) dichloromethane is present as a solvent in said amorphous form in an amount of from 1 ppm to 600 ppm, (II) water is present as a solvent in an amount of from 0.01 wt.-% to 10 wt.-%, and (III) solvents other than dichloromethane and water are present in a total amount of from 0 ppm to 5000 ppm, preferably water is present in an amount of from 0.5 wt.-% to 4.2 wt.-% and solvents other than water are present in the aforementioned amounts.

The telaprevir composition can comprise said amorphous telaprevir and said crystalline form C in a total amount of at least 90% and up to 100%, meaning that 0-10% can be other crystal forms.

The compound of Formula 1 in said telaprevir composition can also have a purity as defined in item 1.1 above, for example a purity in the range of from 95 HPLC area-% to 99.9 HPLC area-%.

4.1 The telaprevir composition of item 4 may consist of (i) at least 70 wt.-% of amorphous telaprevir according to any of items 2-3.6, and (ii) up to 30 wt.-% of crystalline Form C with other crystalline forms optionally being present. It can also comprise 0 to 30 wt.-% of Form C with no other crystal forms being present. It can also comprise 1 to 30 wt.-% of Form C with other crystalline forms being present in an amount up to 1 wt.-%. It can also comprise 5 to 30 wt.-% of Form C with other crystalline forms being present in an amount up to 5 wt.-%. It can also comprise 0 to 20 wt.-% of Form C with no other crystal forms being present. It can also comprise 1 to 20 wt.-% of Form C with other crystalline forms being present in an amount up to 1 wt.-%. It can also comprise 5 to 20 wt.-% of Form C with other crystalline forms being present in an amount up to 5 wt.-%. It can also comprise 0 to 10 wt.-% of Form C with no other crystalline forms being present. It can also comprise 1 to 10 wt.-% of Form C with other crystalline forms being present in an amount up to 1 wt.-%. It can also comprise 5 to 10 wt.-% of crystalline Form C with other crystalline forms being present. The content of Form C can be determined by comparing the amount of Form C by PXRD with standardized synthetic mixtures. Standardized mixtures are prepared by mixing Form C and amorphous telaprevir on a %w/w basis and measuring the intensity of the peak at about 7 degrees 2-theta. A calibration curve obtained in this way can then be used to determine the amount of Form C in amorphous Telaprevir of unknown composition.

4.2 The telaprevir composition of item 4 or item 4.1 may conform with ICH residual solvent guidelines as described in item 3.1.

4.3 The telaprevir composition of any of items 4-4.2 may comprise solvents as e.g. defined in items 3, 3.1 and 4.5

4.4 The telaprevir composition of any of items 4-4.3 can comprise at least 80 wt.-%, at least 90 wt.-%, at least 95 wt.-%, at least 98 wt.-%, of the amorphous form of telaprevir according to the invention, with the remainder preferably being crystalline forms, in particular Form C.

4.5 The telaprevir composition of items 4-4.4, comprising less than 600 ppm of dichloromethane. Within the aforementioned range, dichloromethane can be present in an amount of than less than 400 ppm. Within the aforementioned range, dichloromethane can be present in an amount of less than 350 ppm. Within the aforementioned range, dichloromethane can be present in an amount of less than 300 ppm. Within the aforementioned range, dichloromethane can be present in an amount of less than 250 ppm. Within the aforementioned range, dichloromethane can be present in an amount of less than 200 ppm. Within the aforementioned range, dichloromethane can be present in an amount of less than 150 ppm. Within the aforementioned range, dichloromethane can be present in an amount of even less than 100 ppm of dichloromethane. In the aforementioned ranges, the lower limit can e.g. be 1 ppm. In the aforementioned ranges, the lower limit can e.g. be 10 ppm. In the aforementioned ranges, the lower limit can e.g. be 20 ppm. In the aforementioned ranges, the lower limit can e.g. be 30 ppm. In the aforementioned ranges, the lower limit can e.g. be 40 ppm. In the aforementioned ranges, the lower limit can e.g. be 50 ppm. Within the aforementioned ranges, dichloromethane can be present in an amount of 20 ppm-600 ppm. Within the aforementioned ranges, dichloromethane can be present in an amount of 50 ppm-600 ppm. Within the aforementioned ranges, dichloromethane can be present in an amount of 50 ppm-300 ppm.

4.6 The telaprevir composition of items 4-4.5, wherein dichloromethane is the only organic solvent which is present.

4.7 The telaprevir composition of items 4-4.6, which is polymorphically stable for 2 weeks on exposure to a relative (equilibrium) humidity of 70% (+/−5%) at 50° C. (+/−2° C.), wherein polymorphically stable means that the PXRD of said telaprevir composition does not change.

4.8 The telaprevir composition of items 4-4.7, being in the form of particles, preferably particles consisting of at least 95 wt.-%. Within the aforementioned range, the amount can be at least 97 wt.-%. Within the aforementioned range, the amount can be at least 98 wt.-% of telaprevir. The telaprevir composition preferably consists of 97 wt.-%-99 wt.-% of telaprevir. The telaprevir composition further preferred consists of 98 wt.-%-99 wt.-% of telaprevir.

4.9 The telaprevir composition of items 4-4.8, wherein said particles do not contain pharmaceutical excipients such as polymeric materials.

Crystalline Form C of telaprevir of Formula 1

suitable as intermediate for the preparation of an amorphous form of telaprevir, characterized by an X-ray powder diffraction pattern with peaks at 2-theta angles of about 6.6±0.2 degrees 2theta, 7.0±0.2 degrees 2theta, 8.0±0.2 degrees 2theta, 8.9±0.2 degrees 2theta, 9.4±0.2 degrees 2theta, 17.6±0.2 degrees 2theta, when using Cu-Kα radiation.

Thus, form C of telaprevir can be characterized by showing an X-ray powder diffractogram comprising characteristic peaks at 2-theta angles of about 6.6±0.2°, 7.0±0.2°, 8.0±0.2°, 8.9±0.2°, 9.4±0.2° and 17.6±0.2°. The X-ray powder diffractogram of form C of telaprevir comprises additional characteristic peaks at 2-theta angles of about 4.4±0.2°, 13.4±0.2°, 13.8±0.2°, 16.0±0.2°, 18.0±0.2°, 18.7±0.2° and 19.7±0.2°. A representative diffractogram is displayed in FIG. 2.

Furthermore, Form C of telaprevir can be characterized as being a nonstoichiometric hydrate. E.g. Form C contains about 0.5 moles of water at a relative humidity of about 40%, a water content of about 0.9 moles of water at a relative humidity of about 70%-80%. At a relative humidity of less than 40% water might be released from the solvate retaining the crystal structure.

Form C of telaprevir is a polymorphically stable form e.g. form C is polymorphically stable e.g. when stored at about 70% relative humidity for a period of 40 days.

5.1 The crystalline Form C of telaprevir of item 5, being further defined as in any of items 1-1.1 and 1.5-1.8.

5.2 The crystalline Form C of telaprevir of item 5 or 5.1 can have a solvent content as e.g. defined in item 3, 3.1 with respect to the amorphous form and 4.5 with respect to the telaprevir composition.

6 Process for the preparation of crystalline Form C of telaprevir according to any of item 5-5.2, or as defined in any of items 1-1.1 and 1.5-1.8, said method comprising the steps of:

(i) providing seed crystals of said crystalline Form C of telaprevir;

(ii) providing a highly concentrated residue by evaporation of solvent from a solution of telaprevir in dichloromethane at reduced pressure and at a temperature in the range of from −78° C. to below 5° C., until a semi-solid mass is formed, wherein the dichloromethane used in step (ii) contains water in amounts up to the saturation of dichloromethane with water;

(iii) adding said seed crystals of step (i) to said highly concentrated residue of step (ii) to form a mixture; and

(iv) evaporating solvent from said mixture of step (iii) to obtain said crystalline Form C of telaprevir.

Water can be present in the dichloromethane solvent in concentrations up to the maximum saturation of water in dichloromethane. For example, the solution in step (ii) of the process according to the invention can comprise 0.05 wt.-% to 0.2 wt.-% of water. Preferably, the dichloromethane is saturated with water (at 25° C.).

The highly concentrated residue in step (ii) may be obtained by dissolving telaprevir in dichloromethane and cooling the resulting solution to a temperature of between about 5° C. to −78° C., more preferably to about 5° C. to about −60° C., more preferably to about 0° C. to about −20° C. The solvent can then be evaporated in vacuo or by flushing with a stream of gas e.g. nitrogen. The evaporation can be preformed slowly over a time range of about 2 h to 24 h more preferably over a time period of 3 h to 4 h until a semi-solid mass is formed. Vacuum or gas flow and temperature are adjusted in a way to have a slow evaporation completed within the time range specified above. The amount of dichloromethane in the dissolution step is not critical as long as a solution of telaprevir is obtained before cooling and evaporation. When carrying out this procedure appropriate measures must be taken to guarantee the presence of water in the mixture to allow the crystallization of the novel hydrate of the invention.

Typical water content of water in methylene chloride is from about 0.2 weight % to about 0.05 weight % typically this value may be maintained during the concentration step. Optionally a moist stream of nitrogen is present.

Form C of telaprevir starts to crystallize after addition of the seed crystals and removal of most of the solvent and the crystallization is completed by evaporation of the mixture more or less to dryness. If seed crystals dissolve when added to the highly concentrated residue, no semi-solid mass/highly concentrated residue has yet been achieved and evaporation is carried out until seed crystals do not dissolve. To obtain pure Form C, the highly concentrated residue should not be concentrated to such an extent that telaprevir already spontaneously precipitates in said highly concentrated residue (prior to addition of seed crystals) as amorphous form or crystalline Form A. However, if the desired Form C may contain some minor amounts of amorphous form or Form A, the highly concentrated residue may contain some amorphous form or Form A. The crystals are then collected and dried in vacuo for several hours, e.g. for two to twenty four hours. Drying temperature is not critical, routinely room temperature to about 60° C. is employed.

The term “room temperature” as used herein is understood to mean temperatures of about 15° C. to about 25° C.

6.1 The process of item 6, wherein the seed crystals in step (iii) are added in an amount of from 1 to 5 wt.-% based on the weight of the telaprevir contained in the semi-solid residue.

6.2 The process of any of items 6-6.1, wherein evaporating solvent in step (iv) provides crystalline Form C of telaprevir having a water content of from 0.5 wt.-% to 4.2 wt-% and a dichloromethane content of from 1 to 600 ppm, wherein the content of further solvents can be limited to levels as described above.

7 Crystalline telaprevir form C obtainable or obtained by using a process as defined in any of items 6-6.2.

8 Pharmaceutical composition comprising

(i) a telaprevir composition according to the invention, e.g. described in any of items 4-4.9, or

(ii) an amorphous form of telaprevir according to the invention, e.g. described in any of items 2-3.6;

(iii) crystalline Form C of telaprevir according to the invention, e.g. described in any of items 5-5.2 and 7;

in admixture with one or more pharmaceutically acceptable excipients.

The pharmaceutical composition can have a dichloromethane content of 1-600 ppm based on the content of telaprevir and a total content of further organic solvents of 0-5000 ppm based on the content of telaprevir.

8.1 The pharmaceutical composition of item 8, wherein the telaprevir composition/the amorphous form of telaprevir is in particulate form, wherein the particles comprise at least 95 HPLC area-% of telaprevir; and do not contain polymeric materials.

8.2 The pharmaceutical composition of any of items 8-8.1, which is polymorphically stable on exposure to a relative humidity of 70% at 50° C. or as described in item 3.5. The expression “polymorphically stable” means that no conversion to a crystalline form occurs, as determined by PXRD.

The invention also relates to a process for preparing a pharmaceutical composition as described herein by combining the amorphous form of telaprevir or the telaprevir composition as described herein with pharmaceutically acceptable excipients.

9 Crystalline Form C of telaprevir according to the invention, e.g. as described in any of items 5-5.1 or 7, the amorphous form of telaprevir according to the invention, e.g. described in any of items 2-3.6, or the telaprevir composition according to the invention, e.g. described in any of items 4-4.10 for use as medicament.

10 Crystalline Form C of telaprevir according to the invention, e.g. as described in any of items 5-5.1 or 7, the amorphous form of telaprevir according to the invention, e.g. as described in any of items 2-3.6, the telaprevir composition according to the invention, e.g. as described in any of items 4-4.10, or the pharmaceutical composition according to the invention, e.g. as described in any of items 8-8.2, for use in a method of treating viral infections, such as infections caused by hepatitis C virus.

11 Pharmaceutical dosage form comprising (i) the crystalline Form C of telaprevir according to the invention, e.g. as described in any of items 5-5.1 or 7, (ii) the amorphous form of telaprevir according to the invention, e.g. as described in any of items 2-3.6, (iii) the telaprevir composition according to the invention, e.g. as described in any of items 4-4.10, and/or (iv) the pharmaceutical composition according to the invention, e.g. as described in any of items 8-8.3.

The step of formulating the substantially pure neat amorphous telaprevir into a dosage form may be carried out by applying techniques known in the art. For example, the neat substantially pure amorphous telaprevir composition can be formulated into tablets by using direct compression, granulation processes, spray-coating processes or the like.

The substantially pure neat amorphous telaprevir of the invention may be admixed with at least one inert customary excipient or carrier such as sodium citrate or dicalciumphosphate or fillers or extenders as for example starches, lactose, sucrose, glucose, mannitol and silicic acid, binders, as for example carboxymethylcellulose, alginates gelatin, polyvinylpyrrolidone, sucrose and acacia, humectants, as for example, glycerol, disintegrating agents, as for example, agar,agar, calcium carbonate, starch, alginates, gelatin, certain complex silicates and sodium carbonate, solution retarders as for example paraffin, absorption accelerators, as for example quaternary ammonium compounds, wetting agents, e.g. glycol monostearate, adsorbents, as for example bentonite, lubricants, as for example talc, calcium stearate, magnesium stearate, solid polyethylene glycols, sodium laurylsulfate, opacifying agents, buffering agents, and agents which release the substantially pure amorphous telaprevir in a certain part of the intestinal tract in a delayed manner.

In some preferred embodiments the substantially amorphous neat telaprevir of the invention may be admixed with a polymer or a plurality of polymers including for example cellulose derivatives, e.g. hydroxypropylmethylcellulose, polyvinylpyrrolidones, polyethyleneglycols, polyvinylalcohols, acrylates, such as polymethacrylate, cyclodextrins and copolymers and derivatives thereof, including for example polyvinylpyrolidine-vinylacetate. In some preferred embodiments the polymer or the plurality of polymers are PH dependententeric polymers. Such polymers include cellulose derivatives, e.g. cellulose acetate phthalate, hydroxypropyl methyl cellulose phthalates, hydroxypropyl methyl acetate succinate, hydroxypropyl methyl cellulose acetate, carboxymethylcellulose or a salt thereof, e.g the sodium salt, cellulose acetate trimellitate, hydroxypropylcellulose acetate phthalate, or polymethylacrylates, e.g. Eudragit®S. In some preferred embodiments the polymer or the mixture of polymers is hydroxymethylpropylcellulose in its variety of grades.

In a preferred embodiment a surfactant is included in the mixture of the subtantially pure amorphous telaprevir and a polymer or a mixture of polymers. Suitable surfactants include but are not limited to, sorbitan fatty esters, polyoxyethylene sorbit esters, sodium laurylsulfate, sodium docedylbenzenesulfate, dioctyl sodium sulfosuccinate, sodium stearate, EDTA or vitamin E or tocol derivates.

The mixture of the substantialy pure amorphous telaprevir and the polymer or a plurality of polymers can be directly compressed into an oral dosage form.

Prior to compression the mixture can be blended with an excipient.

An excipient may be selected, for example from one or more of the following classes of excipients: microcrystalline cellulose, starch, lactose, dicalcium phosphate, lubricant, and sugar. Examples of excipients include pregelatinized starch, gelatine, crosscarmellose sodium, crospovidone, silicon dioxide (e.g., colloidal silicon dioxide, e.g., Cabostil), DC-mannitol, microcristalline cellulose (e.g. Avicel, e.g., avicel PH133, Avicel PH102), dibasic calcium phosphate, e.g., anhydrous dibasic calcium phosphate (e.g. granular anhydrous dibasic calcium phosphate, e.g. A-TAB), sodium stearayl fumarate, sodium starch glycolate. Ethanol can also be added to the amorphous form or Form C as excipient in an amount which does not dissolve said amorphous form or Form C.

12 Method of treating a patient by administering an effective amount of (i) the crystalline Form C of telaprevir according to any of items 5-5.1 or 7, of (ii) the amorphous form of telaprevir according to any of items 2-3.6, of (iii) the telaprevir composition according to any of items 4-4.10, and/or of (iv) the pharmaceutical composition according to any of items 8-8.3.

13 Amorphous form of telaprevir of Formula 1 being polymorphically stable on exposure to a relative equilibrium humidity of up to 70%. Preferably, the amorphous form is stable for 14 days/2 weeks at 70% (+/−5%), preferably at temperature of between 0 to 50° C., preferably at 50° C. A test is described in item 3.5 (+/−2° C.).

Herein, the amorphous form of telaprevir is described with respect to the following aspects (1) process for preparing the amorphous form by the process according to the invention starting from crystalline Form C of telaprevir, (2) amorphous form as product or (3) as part of a pharmaceutical composition/dosage form/telaprevir composition. The definition/characterization of the amorphous form of telaprevir as provided with respect to any of the aforementioned aspects can equally also applied in the context of the respective other aspects. The same applies to the definition of crystalline Form C of the invention which is described with respect to the following aspects (1) process for preparing the amorphous form by the process according to the invention starting from crystalline Form C of telaprevir, (2) crystalline Form C as (intermediate)product or (3) as part of a pharmaceutical composition/dosage form/telaprevir composition.

Thus, within the context of the present invention, the above items can be combined to define the embodiment of the present invention. For example, the dichloromethane contents defined in item 4.5, with low dichloromethane contents generally being desired, can be combined with any other items in order to define the dichloromethane contents of the amorphous form/Form C of telaprevir, of the telaprevir composition and of the pharmaceutical composition/dosage form containing same. In addition to the dichloromethane content, the water content of from 0.5 wt.-% to 4.2 wt-% can be used for defining the amorphous form/Form C of telaprevir, the telaprevir composition and pharmaceutical composition/dosage form containing same. In addition, the purity of the compound of Formula 1 of e.g. 97 HPLC area-% to 99.9 HPLC area-% can be used for defining the amorphous form/Form C of telaprevir, the telaprevir composition and pharmaceutical composition/dosage form containing same. In addition, the contents of solvents other than water and dichloromethane as given in item 3, with low contents of said solvents generally being desirable, can be used for defining the amorphous form/Form C of telaprevir, the telaprevir composition and pharmaceutical composition/dosage form containing same.

Particularly disclosed examples of embodiments of the invention are combinations of items as follows:

1+1.1+1.2+1.3+1.4+1.7; 1+1.1+1.2+1.3+1.4; 1+1.1+1.2+1.3; 1+1.1+1.2; 1+1.1; 3+3.1; 3+3.1+3.6; 3+3.2; 3+3.5; 3+3.6; 3+3.2+3.6; 4+4.1+4.8; 4+4.1+4.5; 4+4.2; 4+4.1; 4+4.3; 4+4.5; 4+4.4+4.3; 4+4.6; 4+4.6+4.8; 4+4.6+4.8+4.9; 4+4.6+4.8+4.9+4.5; 5+5.1+5.2; 5+5.1; 5+5.2; 6+6.1; 6+6.2; 6+6.1+6.2; 8+8.1; 8+8.1+8.2.

The following examples describe the present invention in detail, but are not to be construed to be in any way limiting for the present invention.

The X-ray powder diffraction patterns (XRPD) were obtained with a X'Pert PRO diffractometer (PANalytical, Almelo, The Netherlands) equipped with a theta/theta coupled goniometer in transmission geometry, programmable XYZ stage with well plate holder, Cu-Kα1,2 radiation source (wavelength 0.15419 nm) with a focusing mirror, a 0.5° divergence slit, a 0.02° soller slit collimator and a 0.5° anti-scattering slit on the incident beam side, a 2 mm anti-scattering slit, a 0.02° soller slit collimator, a Ni-filter and a solid state PIXcel detector on the diffracted beam side. The patterns were recorded at a tube voltage of 40 kV, tube current of 40 mA, applying a step size of 0.013° 2° with 80 s per step in the angular range of 2° to 40° 2 theta.

| HPLC Apparatus | Agilent 1100 | | | | | |
|---|---|---|---|---|---|---|
| Column | XBridge C18; 4.6 × 150 mm, 3.5 μm; | | | | | |
| | (Waters order-no 186003034) | | | | | |
| Flow rate | 1.1 mL/min | | | | | |
| Temperature | 38° C. | | | | | |
| Detector | Agilent VWL variable wavelength detector, λ = | | | | | |
| | 210 nm (Attenuation 2000 mAU) | | | | | |
| Integrator | DIONEX Laboratory Data System CHROMELEON | | | | | |
| System | gradient | | | | | |
| Stock solution 1 | Dissolve 0.9 g of K₂HPO₄ in 1000 mL of water | | | | | |
| Stock solution 2 | Mix 500 mL of MeOH, 150 mL of EtOH and 350 mL | | | | | |
| | of ISO | | | | | |
| Eluent A | Mix 400 mL of stock solution 1 with 100 mL of stock | | | | | |
| | solution 2 (pH ~9.6) | | | | | |
| Eluent B | Mix 100 mL of stock solution 2 with 400 mL of | | | | | |
| | ACNL | | | | | |
| Solvent | Mix 400 mL of EtOH, 100 mL of water and 100 μL of | | | | | |
| | H₃PO₄ (pH ~3.7) | | | | | |
| Flow rate | 1.1 mL/min | | | | | |
| Oven temperature | 38° C. | | | | | |
| Stop time | 35 min (Post-time 6 min) | | | | | |
| Injection volume | 6.0 μL | | | | | |
| Detection | λ = 210 nm | | | | | |
| | (Attenuation 2000 mAU for Agilent 1100 detectors) | | | | | |
| Autosampler | 5° C. | | | | | |
| | | t (min) | | | | |
| | | 0 | 8 | 25 | 26 | 35 |
| Gradient | % B | 10 | 35 | 80 | 95 | Stop |

Annotation:

The chemicals specified are to be regarded as mere examples. Products from other manufacturers may be used as well if their suitability has been verified.

Evaluation

Integration of all peaks; comparison of retention times of test and reference solution using a laboratory data system.

The moisture sorption isotherms were recorded with a SPS-11 moisture sorption analyzer (MD Messtechnik, Ulm, D). The measurement cycle was started at 30% relative humidity (RH), decreased in 10% steps down to 0% RH, increased up to 90% RH in 10% steps, decreased in 10% steps down to 0% RH and finally increased in 1 step up to 43% RH to determine subsequently the absolute water content by TGA analysis. The equilibrium condition for each step was set to a mass constancy of ±0.005% over 60 min. The temperature was 25±0.1° C.

Fourier transform infrared (FTIR) spectra was recorded with a Bruker IFS 25 spectrometer (Bruker GmbH, Karlsruhe, D) in the spectral range from 4000 to 400 cm-1 with a resolution of 2 cm⁻¹ (64 scans). The sample was prepared on ZnSe disks with a few crystals using the Bruker IR microscope I, with 15×-Cassegrain-objectives (spectral range 4000 to 600 cm⁻¹, resolution 4 cm⁻¹, 100 interferograms per spectrum).

Differential scanning calorimetry (DSC) was performed with a Diamond-DSC (Perkin-Elmer, Norwalk, Ct., USA) using a Pyris 2.0 software. Approximately 1 to 3±0.0005 mg sample (using a UM3 ultramicrobalance, Mettler, Greifensee, CH) was weighed into an Al-Pan (25 μl) and sealed with a cover, which was perforated by a needle. Dry nitrogen was used as the purge gas (purge: 20 ml*min-1).

### REFERENCE EXAMPLE 1

1 g telaprevir was dissolved in 150 mL of tetrahydrofuran at room temperature. The solvent was rapidly removed using a rotary evaporator (water bath temperature 40-45° C.) and vacuum of 300-10 mbar. The solid residue was dried overnight (about 14 h) at room temperature and 20-30 mbar in a drying oven. Yield: 1.0 g. The sample showed the typycal pattern of an amorphous material. Residual Solvent: 1.8% THF (determined by GC).

### REFERENCE EXAMPLE 2

1 g telaprevir was dissolved in 50 mL of dichloromethane at room temperature. The solvent was rapidly removed using a rotary evaporator (water bath temperature 40-45° C.) and vacuum of 300-10 mbar. The solid residue was dried overnight (about 14 h) at room temperature and 20-30 mbar in a drying oven. Yield: 1.0 g. The sample showed the typical pattern of an amorphous material. Residual Solvent: 1.7% dichloromethane (determined by GC).

### EXAMPLE 1

### Preparation of Seeds of Form C of Telaprevir

1000 mg telaprevir form A (#S48) are dissolved under slight stirring in 2 mL dichloromethane to obtain a clear solution, which is filtered through a 0.44 mm syringe-filter into a scintillation vial. The vial (height 4.5 cm, diameter 1.5 cm) is placed in a fridge at 5° C. to evaporate the solvent (approximately 90-95% of the solvent is evaporated). After 24 h the first crystals of form C appear. After complete evaporation the white solid is dried at 60° C. for 2 h.

### EXAMPLE 2

### Preparation of Crystalline Form C of Telaprevir

Telaprevir form A (1 g) was dissolved in dichloromethane (20 mL, water content by KF 0.1%) in a round bottom flask and placed on a rotary evaporator. The water bath was cooled to 0° C. and the pressure in the system was adjusted to 200 mmHg and the evaporation was performed at a flow rate of about 5 mL per hour for about 4 hours. When a gelatinous mass/semi-solid mass became visible seeds form experiment 1 were added and the evaporation was continued for further 2 hours.

Form C was obtained as a white crystalline solid after drying in vacuo (about 20 mbar) at ambient temperature for about 20 hours. The sample was then analysed by PXRD 2-theta values and relative intensities are shown in Table 1.

TABLE 1

| Angle 2 | |
|---|---|
| theta | relative intensity |
| 4.42 | 10.00 |
| 6.59 | 78.00 |
| 6.96 | 100.00 |
| 7.95 | 36.00 |
| 8.88 | 63.00 |
| 9.39 | 65.00 |
| 11.11 | 6.00 |
| 11.48 | 6.00 |
| 12.23 | 6.00 |
| 13.38 | 12.00 |
| 13.94 | 4.00 |
| 14.81 | 21.00 |
| 15.99 | 16.00 |
| 17.61 | 47.00 |
| 18.04 | 30.00 |
| 18.71 | 16.00 |
| 19.74 | 13.00 |
| 21.24 | 6.00 |
| 23.25 | 4.00 |
| 24.13 | 6.00 |
| 26.72 | 3.00 |
| 29.34 | 3.00 |

### EXAMPLE 3

### Preparation of Amorphous Telaprevir

Telaprevir form C (500 mg) was placed in a mortar and gently ground with a pestle into a powder over the course of four minutes. The powder was then analyzed by PXRD. PXRD showed the sample to be amorphous.

### CITED LITERATURE

US 2012/0083441 A1;

WO 2007/022459 A2;

WO 2007/098270 A2;

WO 2008/106151 A2;

WO 2009/032198 A1;

“Die Tablette, Handbuch der Entwicklung, Herstellung and Qualitätssicherung”, Annette Bauer-Brandl, Wolfgang A. Ritschel, third edition, 2012, German language, Editio Cantor Verlag Aulendorf, Chapter 4.8.11, page 419;

K. Fischer Angew. Chemie 48, 394 (1935);

Kwong et al. (Nature Biotechnology 29, 11, 993-1003, 2011);

Turner et al. (Chemical Communications 2010, 46(42), 7918); and

PriorArtDatabase (ip.com, IPCOM000213558D) “Amorphous (1S,3aR,6aS)-N-(1(S)-(2-(Cyclopropylamino)oxalyl)butyl)-2-(N-(pyrazin-2ylcarbonyl)-L-cyclohexylglycyl-3-methyl-Lvalyl)perhydrocyclopenta[c]pyrrole-1-carboxamide”, Dec. 21, 2011.

1. A process for the preparation of an amorphous form of telaprevir of Formula 1 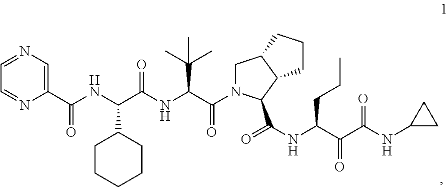 [SMILES: CCC[C@H](NC(=O)[C@@H]1[C@H]2CCC[C@H]2CN1C(=O)[C@@H](NC(=O)[C@@H](NC(=O)c1cnccn1)C1CCCCC1)C(C)(C)C)C(=O)C(=O)NC1CC1]
comprising the steps of:
(i) providing a crystalline Form C of telaprevir having an X-ray powder diffraction pattern with peaks at 2-theta angles of 6.6±0.2 degrees 2theta, 7.0±0.2 degrees 2theta, 8.0±0.2 degrees 2theta, 8.9±0.2 degrees 2theta, 9.4±0.2 degrees 2theta, 17.6±0.2 degrees 2theta, when using Cu-Kα radiation, and
(ii) converting said crystalline Form C of telaprevir into said amorphous form of telaprevir.

2. The process of claim 1, wherein converting said crystalline Form C of telaprevir into said amorphous form of teleprevir in step (ii) is not performed by dissolving or melting said crystalline Form C of telaprevir, such that the desired percentage of amorphous form is obtained.

3. An amorphous form of telaprevir obtainable or obtained by the process according to claim 1, wherein converting said crystalline Form C of telaprevir into said amorphous form of telaprevir is not performed by dissolving/adding solvents or melting said crystalline Form C of telaprevir.

4-7. (canceled)

8. A crystalline Form C of telaprevir of Formula 1 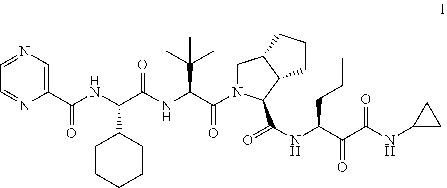 [SMILES: CCC[C@H](NC(=O)[C@@H]1[C@H]2CCC[C@H]2CN1C(=O)[C@@H](NC(=O)[C@@H](NC(=O)c1cnccn1)C1CCCCC1)C(C)(C)C)C(=O)C(=O)NC1CC1]
suitable as intermediate for the preparation of an amorphous form of telaprevir, having an X-ray powder diffraction pattern with peaks at 2-theta angles of 6.6±0.2 degrees 2theta, 7.0±0.2 degrees 2theta, 8.0±0.2 degrees 2theta, 8.9±0.2 degrees 2theta, 9.4±0.2 degrees 2theta, 17.6±0.2 degrees 2theta, when using Cu-Kα radiation.

9. A process for the preparation of crystalline Form C of telaprevir according to claim 8, said method comprising the steps of:
(i) providing seed crystals of said crystalline Form C of telaprevir;
(ii) providing a highly concentrated residue by evaporation of solvent from a solution of telaprevir in dichloromethane at reduced pressure and at a temperature in the range of from −78° C. to below 5° C., until a semi-solid mass is formed, wherein said dichloromethane contains water in amounts up to the saturation of dichloromethane with water;
(iii) adding said seed crystals of step (i) to said highly concentrated residue of step (ii) to form a mixture; and
(iv) evaporating solvent from said mixture of step (iii) to obtain said crystalline Form C of telaprevir.

10. The process of claim 9, wherein the seed crystals in step (iii) are added in an amount of from 1 to 5 wt.-% based on the weight of the telaprevir contained in the semi-solid residue.

11-16. (canceled)

17. The amorphous form of telaprevir according to claim 3, wherein the crystalline Form C, which is used for obtaining the amorphous form of telaprevir, has a water content of up to 4.2 wt.-%.

18. The process of claim 1, wherein converting said crystalline Form C of telaprevir into said amorphous form of teleprevir in step (ii) is not performed by dissolving or melting said crystalline Form C of telaprevir and is performed by grinding at a temperature in the range of from 0° C. to 50° C., such that the desired percentage of amorphous form is obtained.

19. The process of claim 1, wherein converting said crystalline Form C of telaprevir into said amorphous form of teleprevir in step (ii) is not performed by dissolving or melting said crystalline Form C of telaprevir and is performed by grinding at a temperature in the range of from 20° C. to 50° C., such that the desired percentage of amorphous form is obtained.

20. An amorphous form of telaprevir obtainable or obtained by the process according to claim 2, wherein converting said crystalline Form C of telaprevir into said amorphous form of telaprevir is not performed by dissolving/adding solvents or melting said crystalline Form C of telaprevir.
